(19) United States
(12) Patent Application Publication
(10) Pub. No.: US 2010/0249529 A1
(43) Pub. Date: Sep. 30, 2010

(54) Pain Monitoring Apparatus and Methods Thereof

(75) Inventors: Wei-Zen Sun, Taipei City (TW); Jiann-Shing Shieh, Taipei (TW); Chun-Yi Dai, JhongHe City (TW)

Correspondence Address: Jackson Intellectual Property Group PLLC, 106 Starvale Lane, Shipman, VA, 22971, US

(73) Assignee: National Taiwan University, Taipei City (TW)

(21) Appl. No.: 12/483,069

(22) Filed: Jun. 11, 2009

(30) Foreign Application Priority Data
Nov. 13, 2008 (TW) 097143847

(51) Int. Cl. A61B 5/00

(52) U.S. Cl. 600/300

(57) ABSTRACT

A system for monitoring patient control analgesia (PCA) is provided. The system is used with or without a web page at bedside or a remote end. With the system, patients obtain good pain caring; doctors are provided with abundant reference data; and vendors get controls on device logistics.

### FIELD OF THE INVENTION

The present invention relates to monitoring pain; more particularly, relates to a monitoring device which provides real-time patient control analgesia (PCA) data with a complete caring history for a good pain caring quality, a thorough PCA device monitoring and an efficient PCA logistics.

### DESCRIPTION OF THE RELATED ARTS

Pain is a fifth physiological index to the other four indexes: temperature, blood pressure, pulse rate and breath frequency. But this index is quite subjective, not so reliable for clinical use. A drug supplying device for PCA may be used for one time or multiple times to ease a pain, yet it does not read drug-using history for analysis. Statuses of the pain are acquired by asking questions clinically. Hence, although a PCA device may be used, only oral statements of pain are concerned, not history records. In general, pain statuses are obtained during visiting patient while other information concerning the pain are not known; so, some big concern about the pain may be ignored through such a direct and rapid way.

An “i-Pain system” is revealed in IEEE Biomedical Engineering-2007, as shown in FIG. 12, which comprises a data collection layer 31 having devices for collecting data; a data transmitting layer 32 for transmitting data to a remote-end server through the Internet; an intellectual analysis layer 33 for processing an artificial intelligent algorithm with the data in the remote-end server; and a quality monitoring and evaluation layer 34 for supplying data obtained from the intellectual analysis layer 33 for a total quality pain management (TQPM). The system provides PCA data about drug consumption and pain status, and simplifies collecting and storing process of PCA data. But it is usually used for back-end study and does not have a structure for analysis and feedback; and detail descriptions of structural parts are not available. In addition, a pain score is mainly obtained from oral statements of a patient without other supporting data, so the pain score becomes not reliable.

A pain record is obtained at a certain time. Without pain history records of a patient, a big turn to the pain may be thus ignored and a situation of the patient may become worse without exact and correct treatment. In the other hand, every pain is very unique and personal. For example, a first patient may demand drug for 10 times and may feel more painful than a second patient who demands drug for 30 times. In the like, the first patient may press a button 20 times for demanding a drug and may not feel more painful than the second patient who press button 5 times only. It means that drug consumption and demand times are not the only criteria.

In short, because the traditional pain caring is only done with basic data and information obtained on visiting patient yet in short of history data, pain diagnosis become not reliable and pain caring is not effective. Hence, the prior art does not fulfill all users' requests on actual use.

### SUMMARY OF THE INVENTION

The main purpose of the present invention is to obtain a monitoring device which provides real-time PCA data with a complete caring history for a good pain caring quality, a thorough PCA device monitoring and an efficient PCA logistics.

The second purpose of the present invention is to provide added values of PCA for good caring to patients, abundant reference to doctors and devices logistics to vendors.

To achieve the above purposes the present invention is a pain monitoring apparatus and methods thereof, where the pain monitoring apparatus comprises a PCA device, a computer monitoring module, a web monitoring module and a web monitoring module database; the PCA device obtains patient pain data which comprises a patient basic data, a patient bedside information and a patient PCA using history; the computer monitoring module receives the patient pain data from the PCA device through a communication interface; the computer monitoring module stores the patient pain data in a computer monitoring module database to be exchanged, analyzed and integrated; the web monitoring module receives the patient pain data through the Internet to be further analyzed; the web monitoring module database comprises a real-time pain monitoring platform, a PCA machine and drug logistic platform and a patient pain management platform; the web monitoring module database is stored with results of categorizing the analyzed patient pain data by the web monitoring module; and the pain monitoring apparatus has a method used with a web page and a method used without a web page. Accordingly, a novel pain monitoring apparatus and methods thereof are obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the structural view showing the preferred embodiment according to the present invention;

FIG. 2A is the view showing the method used without the web page;

FIG. 2B is the view showing the method used with the web page;

FIG. 3 is the view showing the patient bedside information screen;

FIG. 4 is the view showing the PCA data analysis switching chart;

FIG. 5A is the view showing the demand times chart;

FIG. 5B is the view showing the drug consumption chart;

FIG. 5C is the view showing the FPITT index chart;

FIG. 5D is the view showing the group analysis chart;

FIG. 6A is the view showing the comparison of the demand times charts;

FIG. 6B is the view showing the comparison of the drug consumption charts;

FIG. 6C is the view showing the comparison of the FPITT index charts;

FIG. 7A is the view showing the first PDA interface on visiting patient;

FIG. 7B is the view showing the second PDA interface on visiting patient;

FIG. 8A is the view showing the first UMPC interface on visiting;

FIG. 8B is the view showing the second UMPC interface on visiting;

FIG. 9 is the view showing the portable device having the PCA device;

FIG. 10 is the view showing the interface of the real-time pain monitoring platform;

FIG. 11 is the view showing the personal pain diary; and

FIG. 12 is the structural view of the prior art.

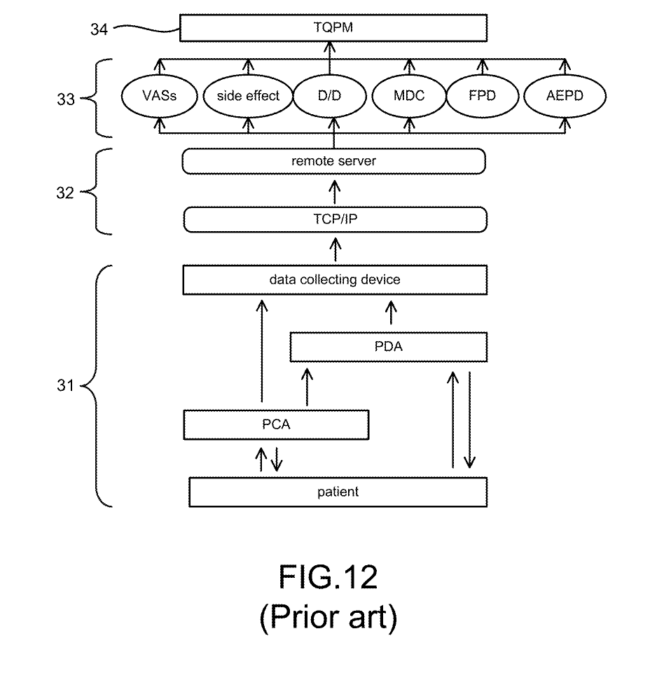

### DESCRIPTION OF THE PREFERRED EMBODIMENT

The following description of the preferred embodiment is provided to understand the features and the structures of the present invention.

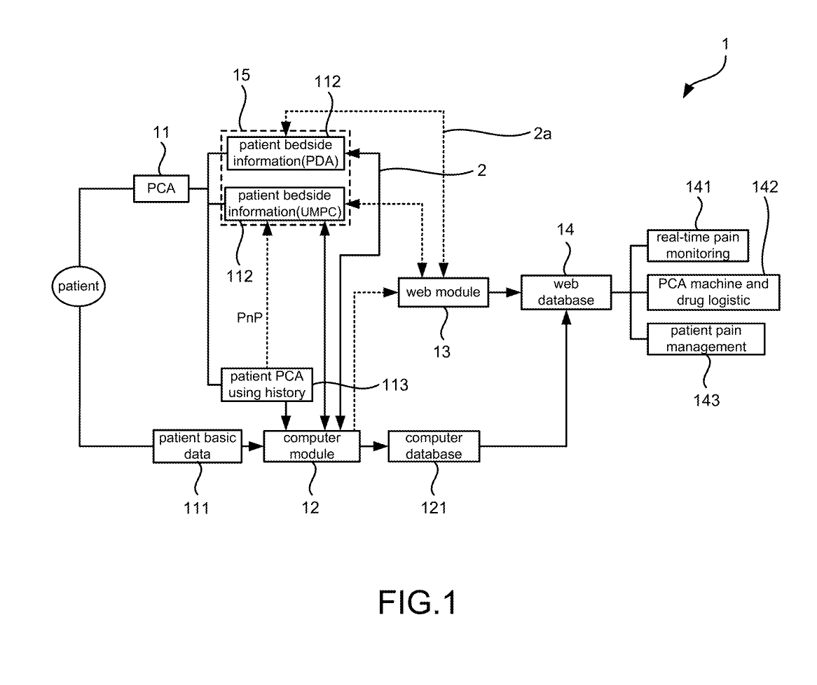

Please refer to FIG. 1, which is a structural view showing a preferred embodiment according to the present invention. As shown in the figure, the present invention is a pain monitoring apparatus and methods thereof. The pain monitoring apparatus 1 comprises a PCA device 11, a computer monitoring module 12, a web monitoring module 13 and a web monitoring module database 14, where patient pain data are collected through a wire connection 2 or a wireless connection 2a to be transmitted to a remote end with a communication interface to obtain analysis and feedback through data analysis to be viewed by doctors, researchers, PCA device providers, expendable providers and patients.

The PCA device 11 generates patient pain data, including patient basic data 111, patient bedside information 112 and a patient PCA using history 113, where the patient basic data 111 comprises a height, a sex, a weight, a description and a safety class of a patient; the patient bedside information 112 comprises a pain score and a side-effect evaluation; and the patient PCA using history 113 comprises a drug using timing, a drug dosage, a demand times and a PCA machine status.

The computer monitoring module 12 obtains the patient pain data from the PCA device 11 through a wire connection with a communication interface and stores the patient pain data in a computer monitoring module database 121 to be exchanged, analyzed and integrated, where the computer monitoring module 12 is a desktop computer.

The web monitoring module 13 obtains the patient pain data through the Internet for analysis.

The web monitoring module database 14 comprises a real-time pain monitoring platform 141, a PCA machine and drug logistic platform 142 and a patient pain management platform 143; and is stored with results of categorizing the analyzed patient pain data by the web monitoring module 13.

The pain monitoring apparatus 1 may further comprise a portable device 15 to obtain the patient pain data from the PCA device 11 through a wireless connection 2a with a PCA blue tooth wireless communication module; and the patient pain data are transmitted to the computer monitoring module 12 or the web monitoring module 13 in a wire or wireless way for data integration, where the PCA blue tooth wireless communication module is a plug-and-play (PnP) device. Thus, a novel pain monitoring apparatus 1 is obtained.

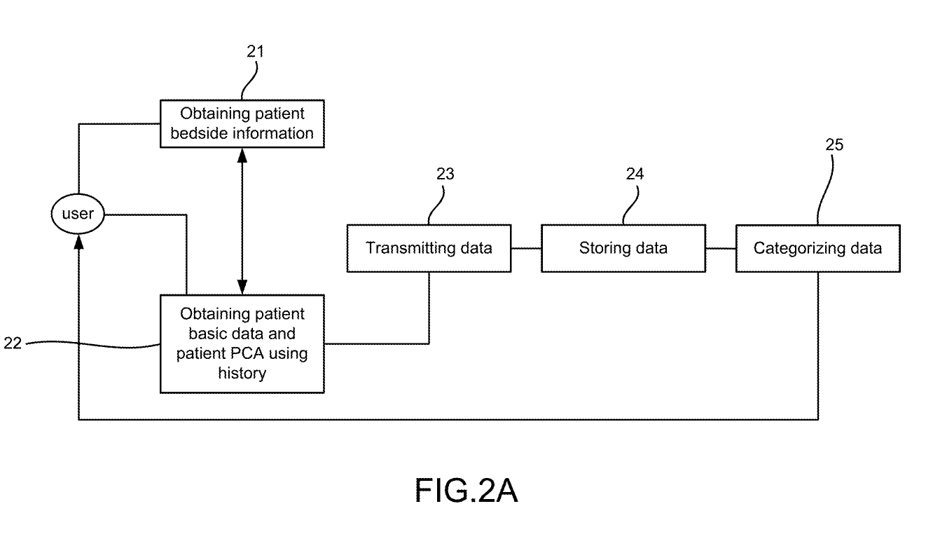

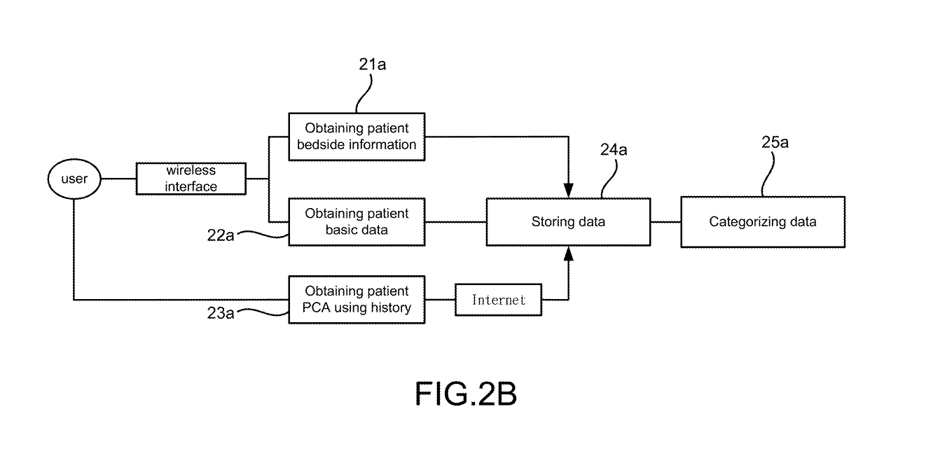

Please further refer to FIG. 2A and FIG. 2B, which are views showing a method used without a web page and a method used with a web page. As shown in the figures, the PCA device 11 is used after an operation by a patient. Patient pain data, including a patient basic data 111, a patient bedside information 112 and a patient PCA using history 113, is generated to be stored in a memory of the PCA device 11. The computer monitoring module 12 stores the patient basic data 111 and the patient bedside information 112 in the computer monitoring module database 121. An LPT interface of the PCA device 11 is transformed into an RS-232 interface for storing the patient PCA using history 113 through a wire connection 2 or a wireless connection 2a.

In FIG. 2A, the present invention has a method used without a web page, comprising the following steps:

(a1) Obtaining patient bedside information 21: The patient bedside information 112 is obtained from the PCA device 11 by the portable device 15 through visiting a patient at bed side.

(b1) Obtaining patient basic data and patient PCA using history 22: The patient basic data 111 and the patient PCA using history 113 are obtained from the PCA device 11 by the computer monitoring module 12 to exchange data with the portable device 15 for processing data analysis; and results of the data analysis are stored in the computer monitoring module database 121.

(c1) Transmitting data 23: The patient basic data 111, the patient bedside information 112 and the patient PCA using history 113 are transmitted through the Internet.

(d1) Storing data 24: The patient basic data 111, the patient bedside information 112 and the patient PCA using history 113 are received by the web monitoring module 13 and are stored in the web monitoring module database 14.

(e1) Categorizing data 25: The analyzed patient basic data 111, the analyzed patient bedside information 112 and the analyzed patient PCA using history 113 are categorized to be stored in the real-time pain monitoring platform 141, the PCA machine and drug logistic platform 142 and the patient pain management platform 143 for different viewers.

In step (a1), a PCA blue tooth wireless communication module is used to process an integrated diagnosis by examining the patient PCA using history 113; and is used to exchange data with the computer monitoring module 12.

In FIG. 2A, the present invention has a method used with a web page, comprising the following steps:

(a2) Obtaining patient bedside information 21a: The patient bedside information 112 is obtained through a wireless network by the web monitoring module 13 at bed side.

(b2) Obtaining patient basic data 22a: The patient basic data 111 is obtained through a wireless network by the web monitoring module 13 at bed side at a nursing station.

(c2) Obtaining patient PCA using history 23a: The patient PCA using history 113 is directly obtained from the PCA device 11 by the computer monitoring module 12 and is transmitted to the web monitoring module 13 through the Internet.

(d2) Storing data 24a: The patient PCA using history 113 is transmitted to the web monitoring module 13 through the Internet. Thus, the patient basic data 111, the patient bedside information 112 and the patient PCA using history 113 are received by the web monitoring module 13 to be stored in the web monitoring module database 14.

(e2) Categorizing data 25: The analyzed patient basic data 111, the analyzed patient bedside information 112 and the analyzed patient PCA using history 113 are categorized to be stored in the real-time pain monitoring platform 141, the PCA machine and drug logistic platform 142 and the patient pain management platform 143 for different viewers.

In the above steps, all data are transmitted to the web monitoring module database 14 through the Internet; yet, data are stored in the computer monitoring module database while corresponding devices do not have the same communication interface.

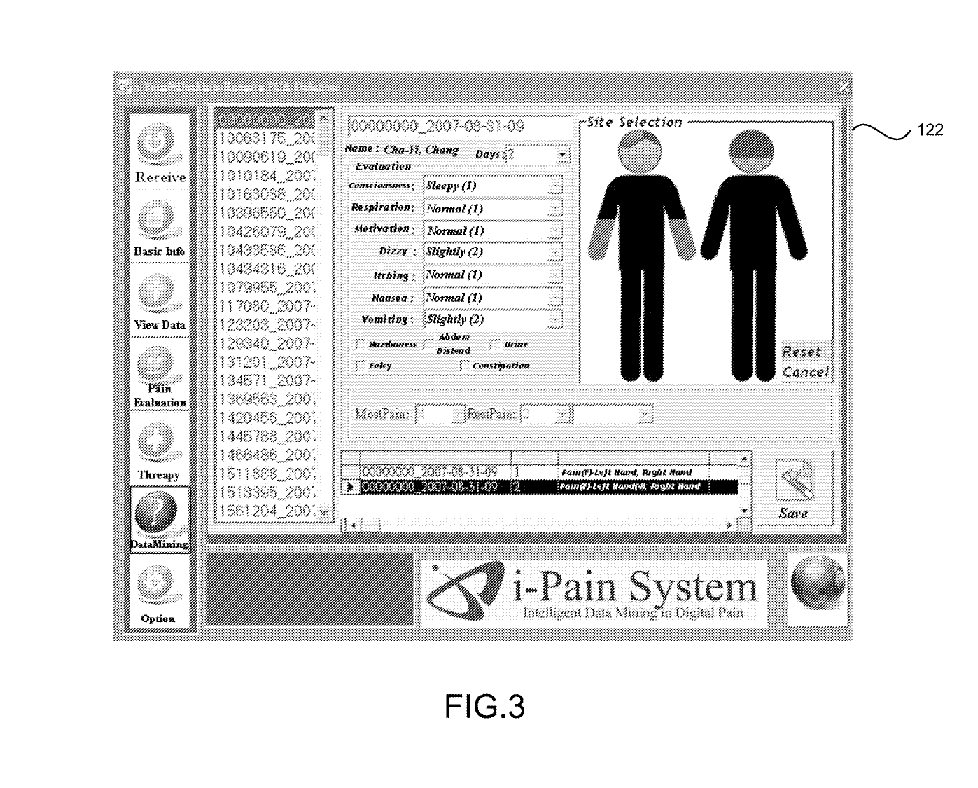

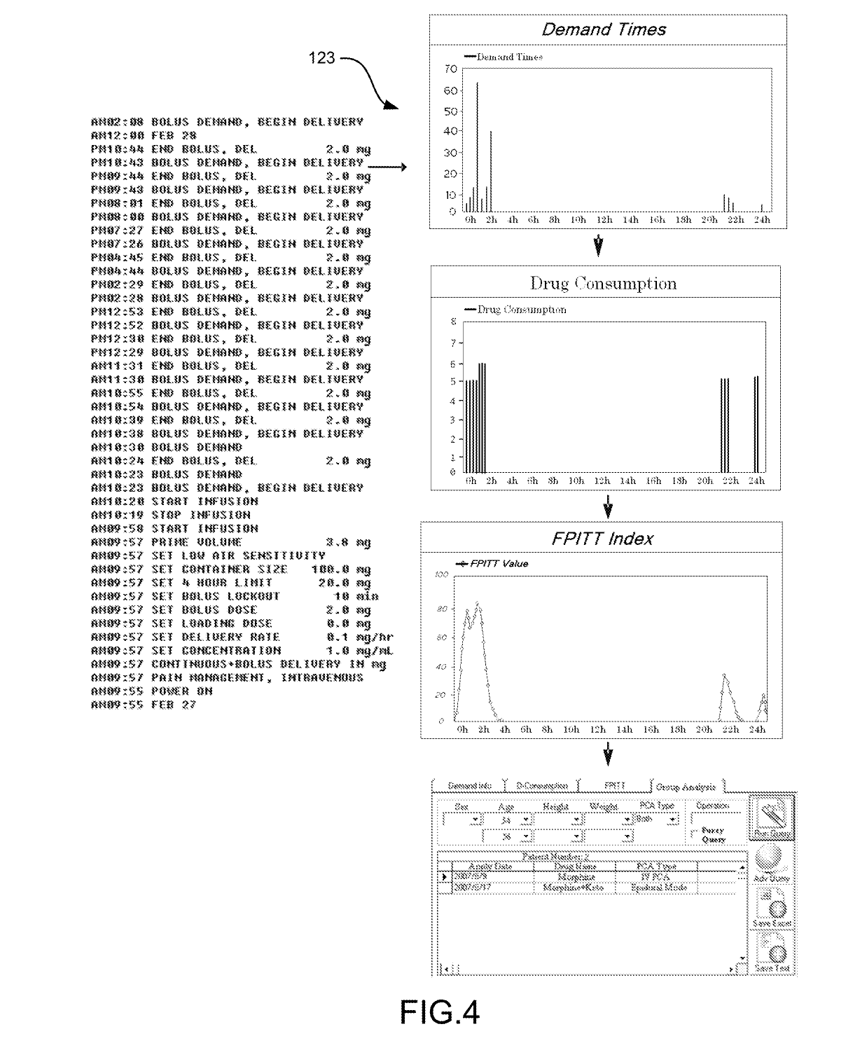

Please refer to FIG. 3 to FIG. 5D, which are views showing a patient bedside information screen, a PCA data analysis switching chart, a demand times chart, a drug consumption chart, a fuzzy pain-intend-to-treat (FPITT) index chart and a group analysis chart. As shown in the figures, a computer monitoring module 12 of the present invention has a clinical visit system; and the computer monitoring module 12 further has a patient bedside information screen.

On the patient bedside information screen, there are functions of data reception, basic data management, pain examining, pain evaluation, dosing records, data analysis and options. Take the pain evaluation as an example: In FIG. 3, a visualized pain evaluation screen 122 shows a medical record number, a number of days for pain, a status evaluation, a visual analogue score (VAS) of pain and a pain position diagram. On the screen, clinical visit data and side effect profile are inputted through the computer monitoring module 12; a status evaluation and a pain position diagram are shown; and all data are integrated into a computer monitoring module database of the computer monitoring module 12.

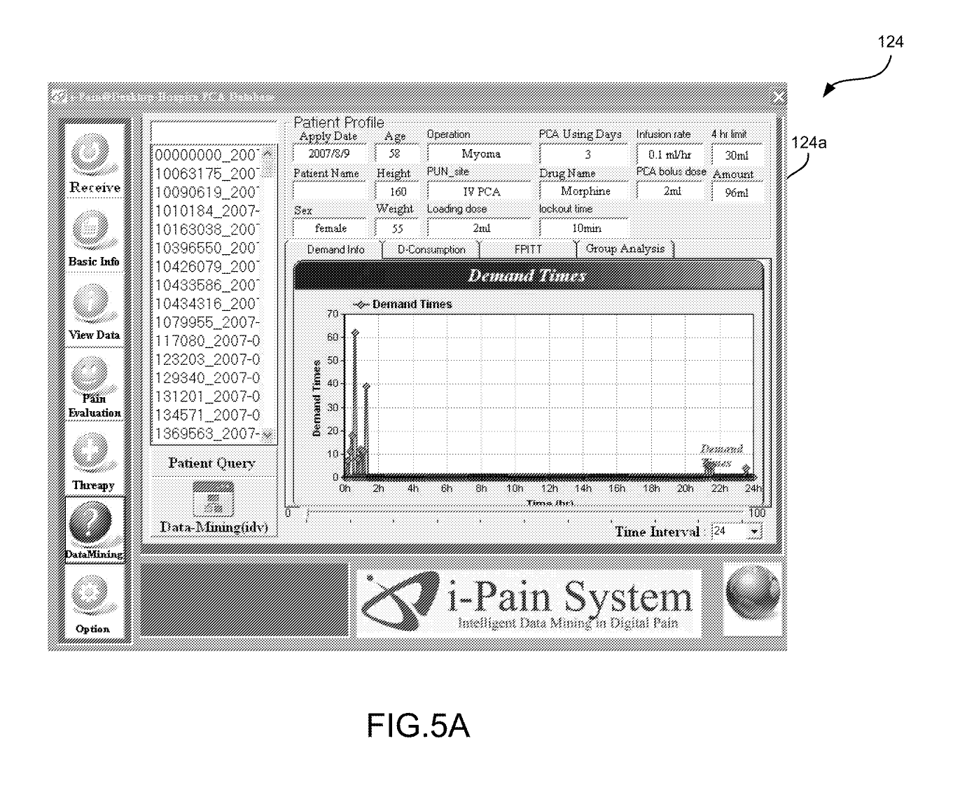

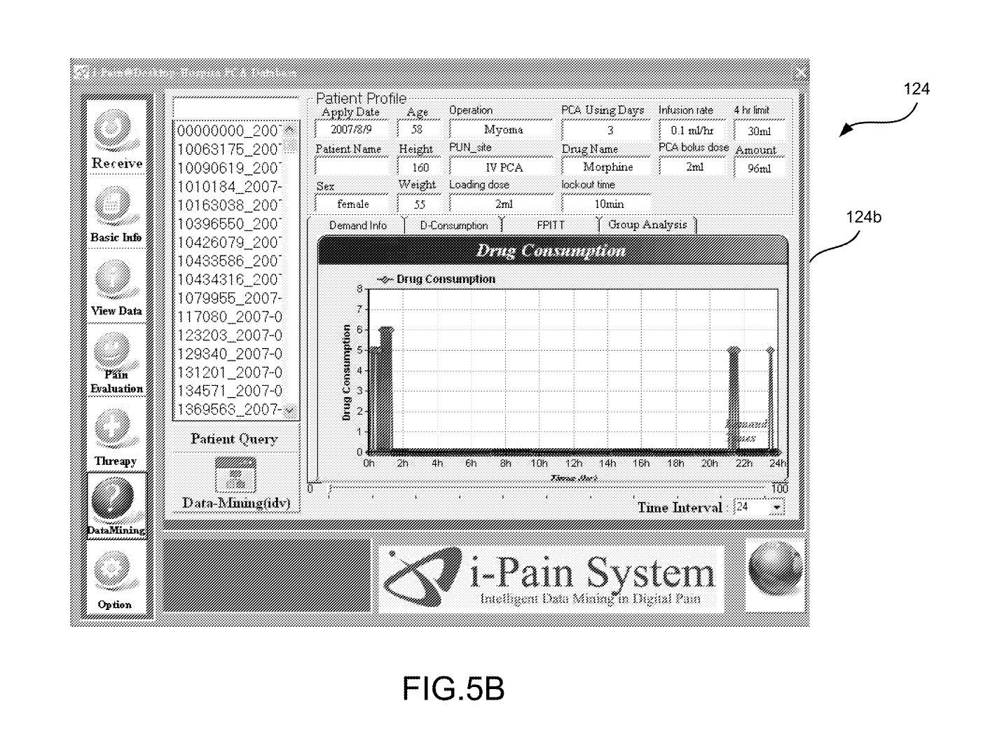

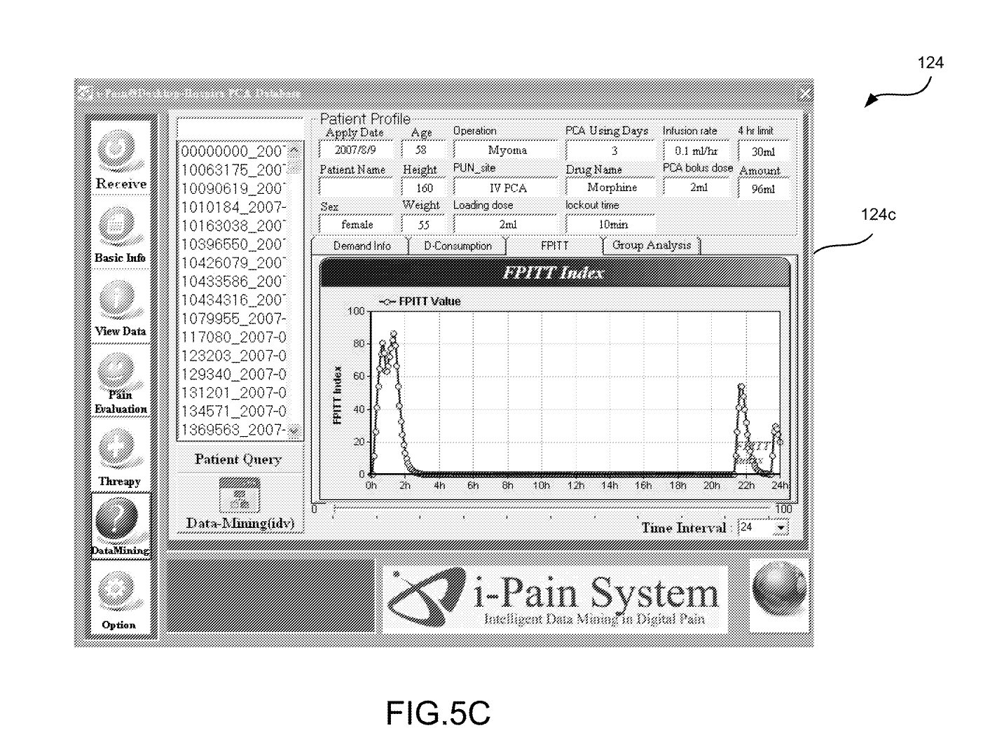

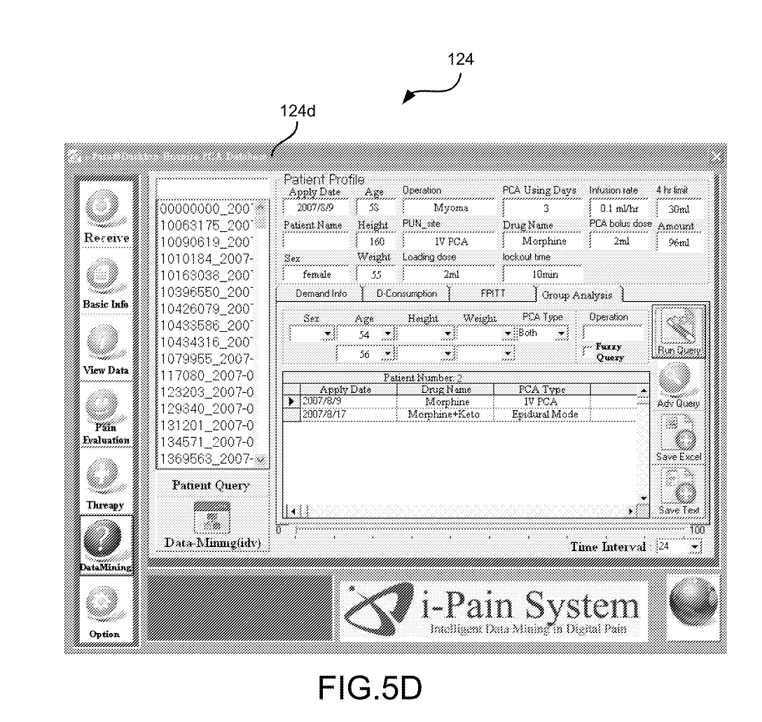

Take the data analysis as an example: In FIG. 3, a PCA data analysis switching chart 123 shows a PCA using history and charts derived from records in the history. All events in the history are collected in a wire or wireless way and show with a list or derived charts. Thus, with the computer monitoring module, a specific patient or group of patient can be filtered out by a structured query language (SQL) statement; then, record or records of patient basic data, patient bedside information and patient PCA using history are combined and outputted into an Excel file as a reference for clinical members. Therein, the present invention has an artificial intelligent algorithm for transforming a text file of PCA data to be outputted into a list and further into visualized charts. In FIG. 5A to FIG. 5D, a demand times chart 124a, a drug consumption chart 124b and a FPITT Index chart 124c are shown on a patient analysis screen 124 and a group analysis chart 124d is further shown on the patient analysis screen 124 after analysis.

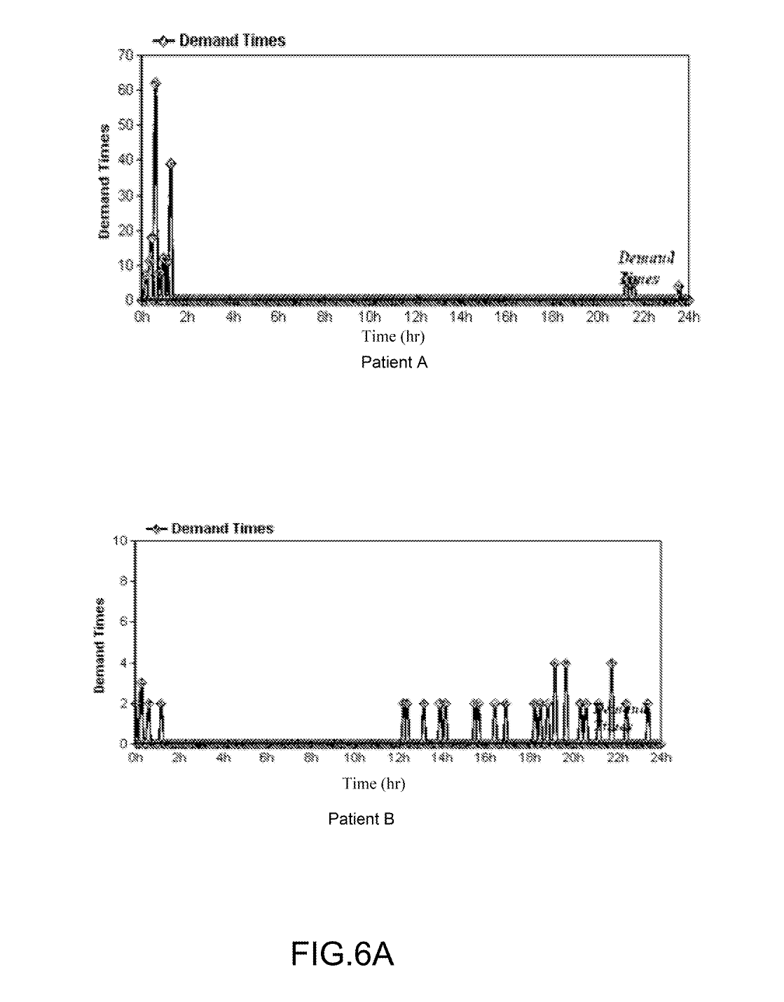

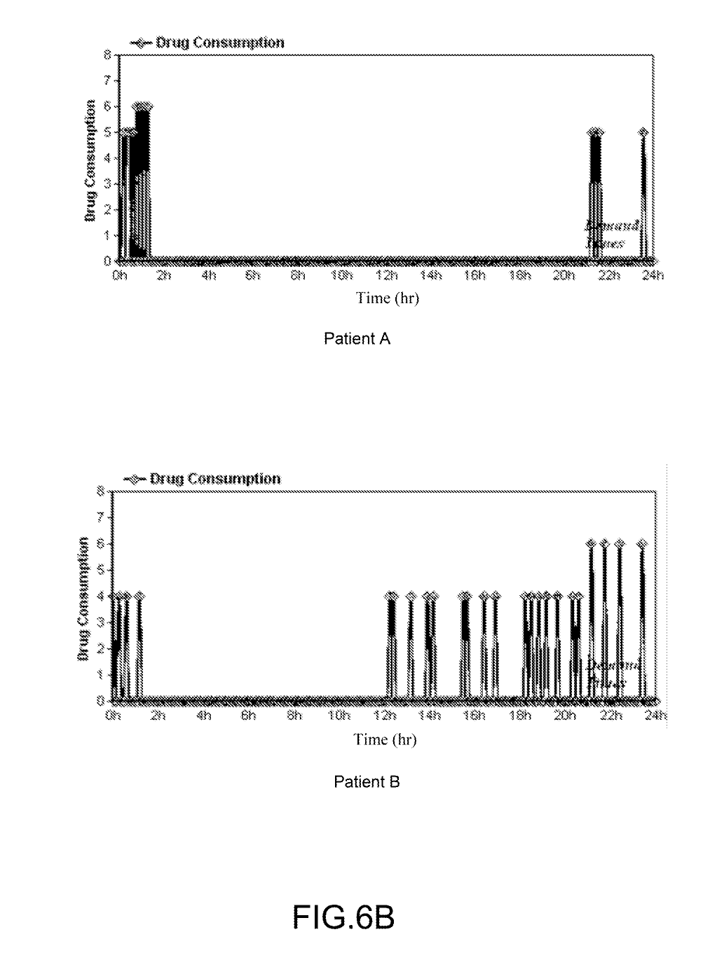

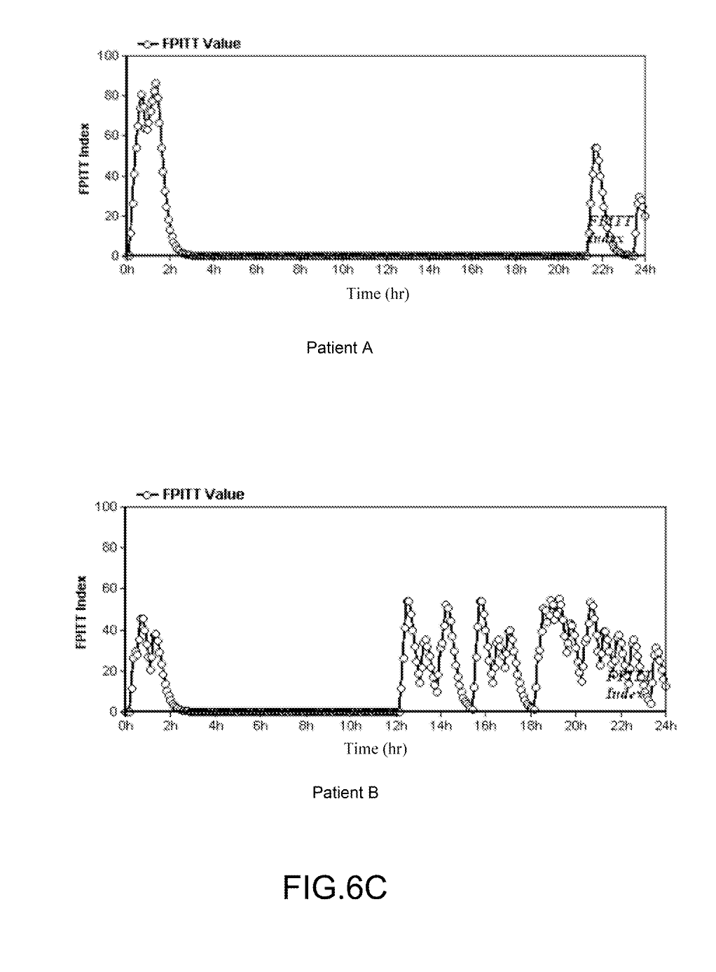

Please refer to FIG. 6A to FIG. 6C, which are views showing comparisons of demand times charts, drug consumption charts and FPITT index charts. As shown in the figures, an artificial intelligent (AI) algorithm is developed by integrating a fuzzy model theory, changeable fuzzy rules and a price tendency analyzing method usually used for financial market; and the AI algorithm is used to obtain a fuzzy pain-intend-to-treat (FPITT) index. The FPITT index is a number between 0 and 100 for pain evaluation, where a FPITT index higher than 60 means a pain hard to be eased by a PCA service. For example, as shown in FIG. 6A, a first patient has a highest demand times more than 60 while a second patient's highest demand times is 4; as shown in FIG. 6B, the first patient has a drug consumption more than that of the second patient; and, as shown in FIG. 6C, the first patient has a safe FPITT index threshold more than 60 while the second patient's FPITT indexes are all in danger. Conclusively, the first patient requires more drugs to ease pain within 0 to 2 hours; and, it is recommended to apply more drugs since some FPITT indexes of the first patient are over or close to a dangerous value. In the other hand, although the second patient takes more drugs than the first patient, no change is required since all FPITT indexes of the second patient are below the dangerous value.

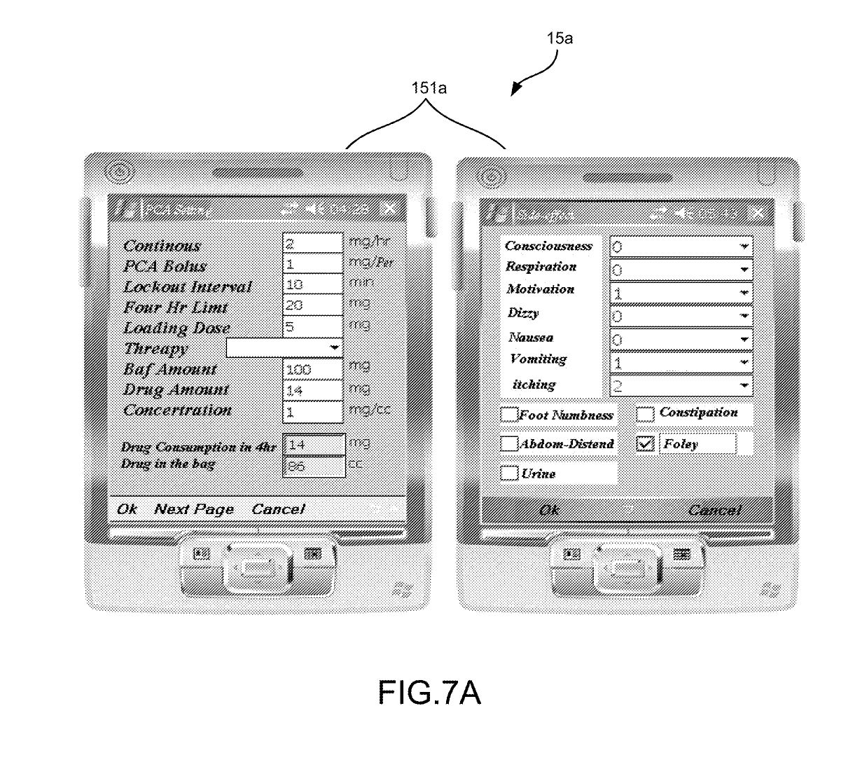

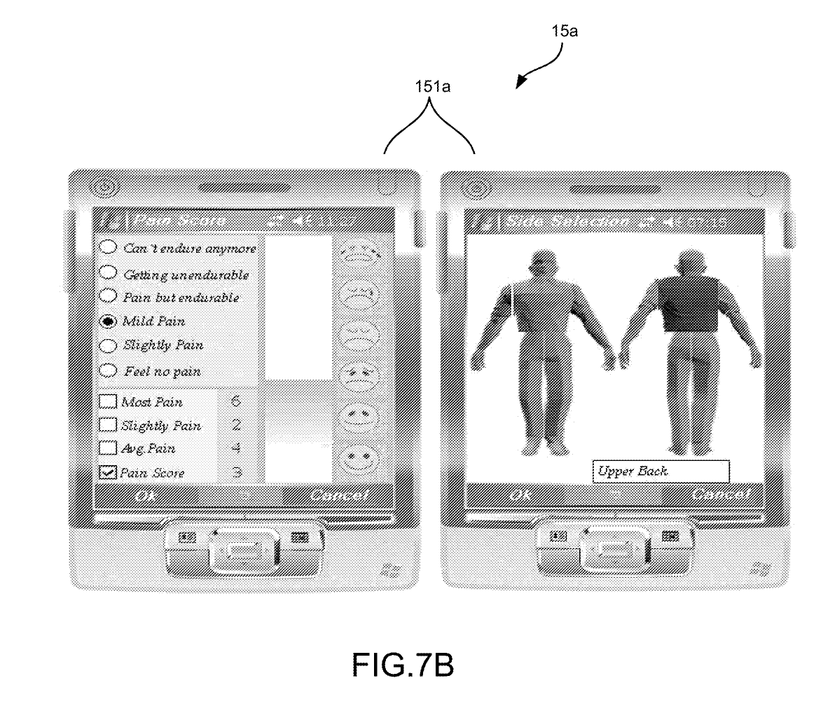

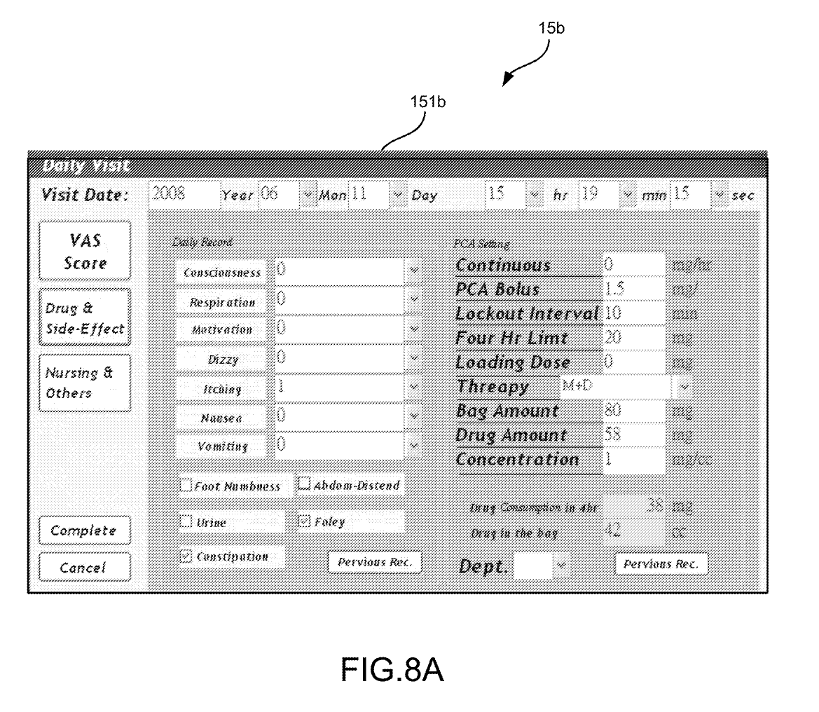

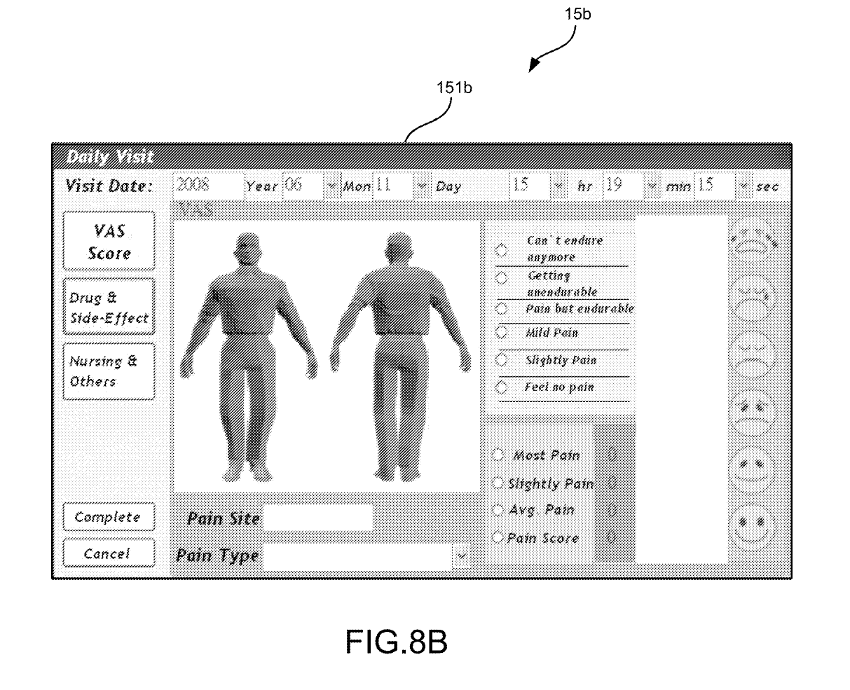

Please refer to FIG. 7A to FIG. 8B, which are views showing a first and a second PDA interfaces on visiting a patient; and views showing a first and a second UMPC interfaces on visiting patient. As shown in the figures, the present invention further uses a portable device on visiting patient, where the portable device can be a personal digital assistant (PDA) 15a or an ultra mobile personal computer (UMPC) 15b, where an electrical questionnaire is built in the PDA 15a and the UMPC 15b. However, the UMPC 15b is more powerful; and, hence, is more capable of diagnosing pain of a patient and integrating real-time pain information.

In FIG. 7A and FIG. 7B, a PDA 15a has a first interface 151a for visiting patient; and, an UMPC 15b having a second interface 151b is used in FIG. 8A and FIG. 8B. All recording interface are graphical for being easily used by disabled person. After visiting patient, data are transmitted back to a nursing station or a web monitoring module in a wire or wireless way for data integration.

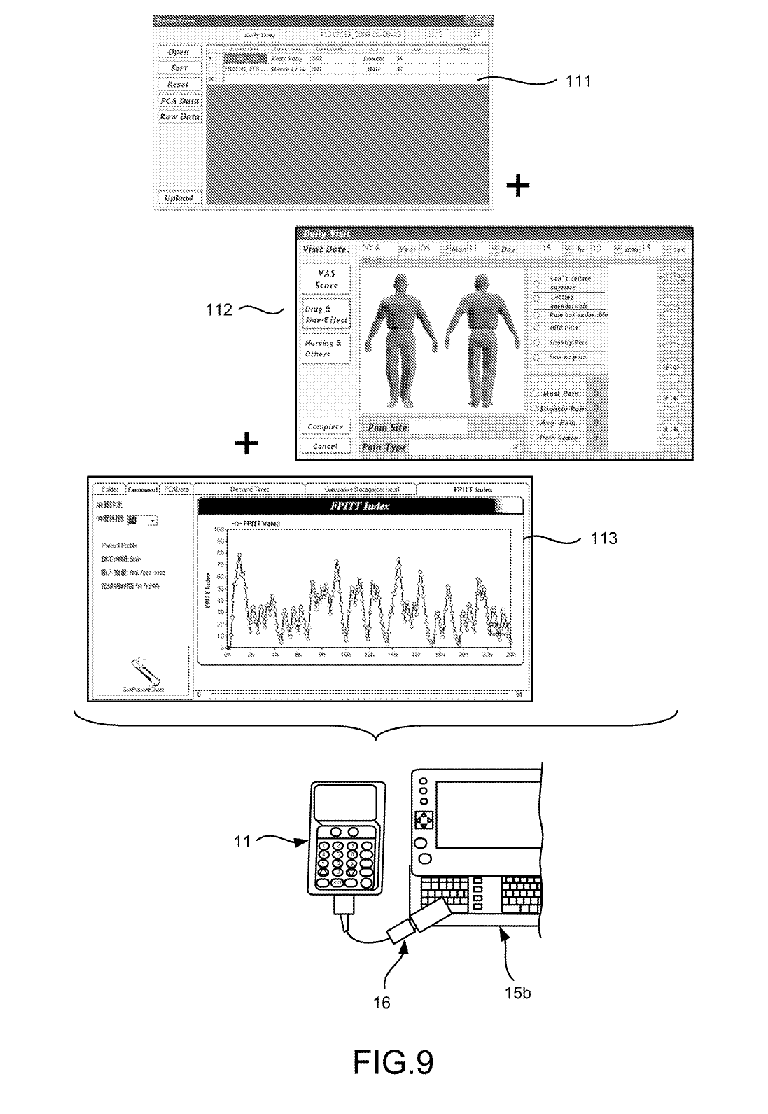

Please refer to FIG. 1 and FIG. 9, which are a structural view showing a preferred embodiment according to the present invention and a view showing a portable device having a PCA device. As shown in the figures, the present invention prefers using a UMPC 15b with a PCA blue tooth wireless communication module 16 for its powerful capability and for being equipped with a data analysis tool, where the PCA blue tooth wireless communication module 16 has a specific wire for transmission and a blue tooth transmission circuit. On using the present invention, a PCA device 11 transmits data to a computer monitoring module 12 through the PCA blue tooth wireless communication module 16 in a wireless way. Thus, by integrating patient basic data 111, patient bedside information 112 and a patient PCA using history 113, all pain information of a patient are obtained for precise diagnosing and treatment.

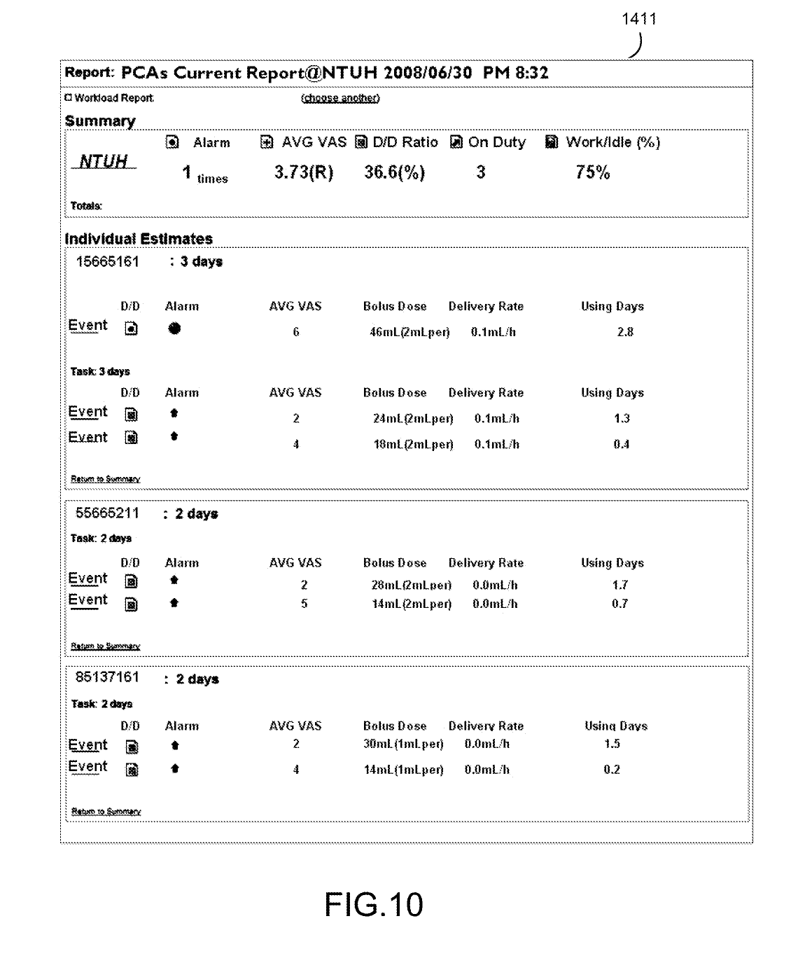

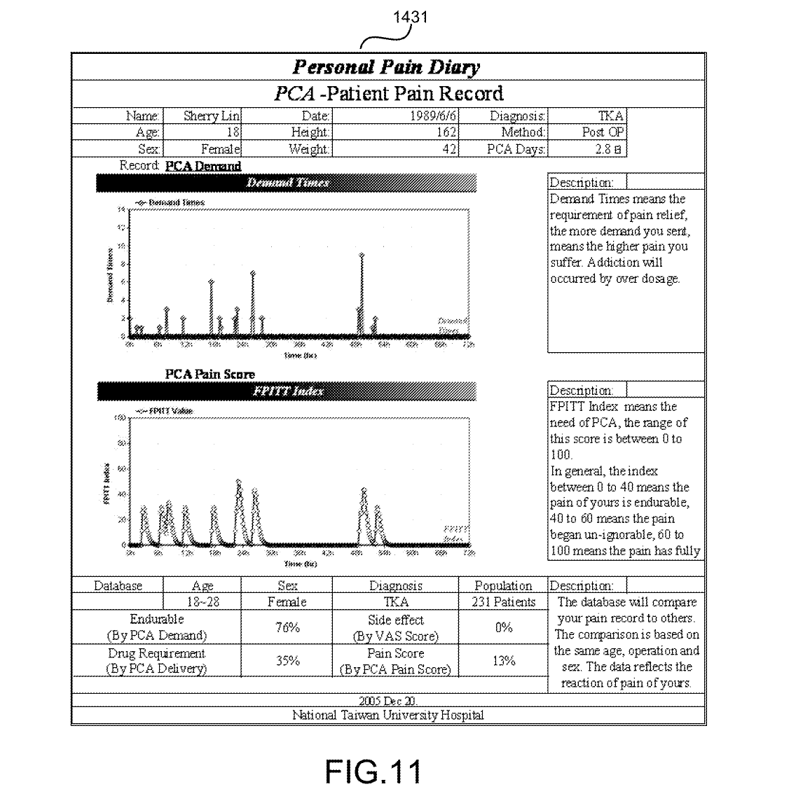

Please refer to FIG. 10 and FIG. 11, which are a view showing an interface of a real-time pain monitoring platform and a view showing a personal pain diary. As shown in the figures, after patient basic data, patient bedside information and a patient PCA using history are transmitted to a web monitoring module database of a web monitoring module, the web monitoring module runs to provide data to a real-time pain monitoring platform, a PCA machine and drug logistic platform and a patient pain management platform.

Firstly, in FIG. 10, a data interface 1411 of the real-time pain monitoring platform shows a total PCA device number, a using PCA device number, VAS values, using days, and data and alarm obtained from the web monitoring module database. Therein, the alarm is used to indicate instability of a patient, which is obtained from a big VAS value or other big values of demand times, drug consumption and FPITT Indexes. Thus, because all data are transferred to the database at remote end, pain statuses are obtained at real time at nursing station. Besides, all data are integrated for improving pain caring quality through analysis.

Secondly, statistical data are obtained from the PCA machine and drug logistic platform through analyzing the three data transferred to the web monitoring module database. Therein, a precise treatment and PCA setups and an effect of a new drug for pain caring are obtained.

Thirdly, in FIG. 13, a pain report interface 1431 is obtained from the patient pain management platform to show a pain caring history. Therein, a group of patients having the similar pain situation are found out for comparison to evaluate the effect of the pain caring.

Thus, with the present invention, excellent pain caring effects for patients are obtained; various PCA parameters for various pains are setup; and PCA devices are monitored for their usages and availabilities.

To sum up, the present invention is a pain monitoring apparatus and methods thereof, where patient pain data are collected from a computer monitoring module to be exchanged, analyzed and integrated to be transmitted to a web monitoring module for a further data analysis process with results presented through a web page for various interactions; and a good pain caring quality, a complete caring history, a thorough PCA device monitoring and an efficient PCA logistics are thus obtained with low cost.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A pain monitoring apparatus, comprising
a patient control analgesia (PCA) device, said PCA device obtaining patient pain data, said patient pain data comprising patient basic data, patient bedside information and a patient PCA using history;
a computer monitoring module, said computer monitoring module obtaining said patient pain data from said PCA device through a communication interface, said computer monitoring module storing said patient pain data in a computer monitoring module database to be exchanged, analyzed and integrated;
a web monitoring module, said web monitoring module obtaining said patient pain data through the Internet to be analyzed; and
a web monitoring module database, said web monitoring module database comprising a real-time pain monitoring platform, a PCA machine and drug logistic platform and a patient pain management platform, said web monitoring module database being stored with results of categorizing said analyzed patient pain data by said web monitoring module.

2. The apparatus according to claim 1,
wherein said computer monitoring module has an artificial intelligent algorithm to obtain a fuzzy-pain-intend-to-treat (FPITT) Index.

3. The apparatus according to claim 1,
wherein said patient basic data comprises a height, a sex, a weight, a description and a safety class.

4. The apparatus according to claim 1,
wherein said patient bedside information comprises a pain score and a side-effect evaluation.

5. The apparatus according to claim 1,
wherein said patient PCA using history comprises a drug using timings, a drug dosage, demand times and a PCA machine status.

6. The apparatus according to claim 1,
wherein said computer monitoring module is a desktop computer.

7. The apparatus according to claim 1,
wherein said pain monitoring apparatus further comprises a portable device to obtain said patient pain data from said PCA device through a wireless connection with a PCA blue tooth wireless communication module.

8. The apparatus according to claim 7,
wherein said portable device is selected from a group consisting of a personal digital assistant (PDA) and an ultra mobile personal computer (UMPC).

9. The apparatus according to claim 7,
wherein said PCA blue tooth wireless communication module is a plug-and-play (PnP) device.

10. The apparatus according to claim 1,
wherein said computer monitoring module is built-in with a clinical visit system.

11. The apparatus according to claim 1,
wherein said pain monitoring apparatus has a method used without a web page, comprising steps of:
(a1) obtaining said patient bedside information from said PCA device by said portable device through visiting a patient at bed side;
(b1) obtaining said patient basic data and said patient PCA using history from said PCA device by said computer monitoring module to exchange data with said portable device to process data analysis and storing results of said data analysis in said computer monitoring module database;
(c1) transmitting said patient basic data, said patient bedside information and said patient PCA using history through the Internet;
(d1) receiving said patient basic data, said patient bedside information and said patient PCA using history by said web monitoring module and storing said patient basic data, said patient bedside information and said patient PCA using history in said web monitoring module database; and
(e1) categorizing said analyzed patient basic data, said analyzed patient bedside information and said analyzed patient PCA using history to be stored in said real-time pain monitoring platform, said PCA machine and drug logistic platform and said patient pain management platform of said web monitoring module database.

12. The apparatus according to claim 11,
wherein, in step (a1), a PCA blue tooth wireless communication module is used to process an integrated diagnosis by examining said patient PCA using history.

13. The apparatus according to claim
wherein said pain monitoring apparatus has a method used with a web page, comprising steps of:
(a2) obtaining said patient bedside information through the Internet by said web monitoring module at bed side of a patient;
(b2) obtaining said patient basic data through the Internet by said web monitoring module at a nursing station;
(c2) obtaining said patient PCA using history by said computer monitoring module;
(d2) transmitting said patient PCA using history to be received by said web monitoring module through the Internet and analyzing said patient basic data, said patient bedside information and said patient PCA using history by said web monitoring module; and
(e1) categorizing said analyzed patient basic data, said analyzed patient bedside information and said analyzed patient PCA using history to be stored in said real-time pain monitoring platform, said PCA machine and drug logistic platform and said patient pain management platform of said web monitoring module database.
